(19) United States
(12) Patent Application Publication
(10) Pub. No.: US 2021/0114385 A1
(43) Pub. Date: Apr. 22, 2021

(54) METHOD FOR MAKING EASY-TO-VIEW CT SHEET

(71) Applicant: Hefei University of Technology, Anhui (CN)

(72) Inventors: Xiaojia Wang, Anhui (CN); Kuo Du, Anhui (CN); Keyu Zhu, Anhui (CN); Sheng Xu, Anhui (CN)

(73) Assignee: Hefei University of Technology, Anhui (CN)

(21) Appl. No.: 17/074,810

(22) Filed: Oct. 20, 2020

(30) Foreign Application Priority Data
Oct. 22, 2019 (CN) 201911006686.4

(51) Int. Cl. B41J 13/00; A61B 6/03; A61B 6/00; H04N 5/32

(52) U.S. Cl. CPC B41J 13/0009; A61B 6/032; A61B 6/4208; A61B 6/54; A61B 6/56; A61B 6/461; H04N 5/32

(57) ABSTRACT

The present invention discloses a method for making an easy-to-view computed tomography (CT) sheet. The method is implemented by the following modules, including an X-ray tomographic scanning device, a computer system, an operating system, and a printing system. The X-ray tomographic scanning device is composed of an X-ray generator, an X-ray tube, and an X-ray detector. The X-ray generator of the X-ray tomographic scanning device is used for X-ray generation. Through increasing detection accuracy of the detector inside the scanning device, radiation dose can be effectively reduced, thereby avoiding large radiation hazards to a patient during scanning. In this way, image quality is improved without increasing the radiation dose or even when the radiation dose is reduced. In addition, a processing method for image reconstruction is further improved under joint action of multiple groups of X-rays, thereby further improving data generation.

### TECHNICAL FIELD

The present invention relates to the field of technologies related to computed tomography (CT) systems, and specifically relates to a method for making an easy-to-view CT sheet.

### BACKGROUND

As medical imaging continues to evolve, medical imaging technology is widely applied to clinical judgment and treatment. Common medical imaging equipment includes CT systems and ultrasound systems. CT is a procedure of scanning with X-rays a layer with a particular thickness of a specific part of a human body. Because different human tissues vary in capability of absorbing X-rays, a computer can be used to reconstruct a cross-sectional image. With regard to psychology, a CT system is used for checking perception, thinking, emotion, willpower behaviors, sleep, psychological development, lies, interpersonal relationship, learning ability, personal style, marriage and family problems, psychological defense mechanisms, physical and mental diseases and the like, thereby comprehensively measuring mental health and psychological quality.

The technology of an existing method for making a CT sheet has the following problems: In the existing method for making a CT sheet, a large amount of radiation dose generally needs to be added for scanning. Consequently, a patient may be affected by radiation during scanning, which causes certain harm to the patient's body. However, if insufficient radiation dose is added, a result of the scanning may be inaccurate.

### SUMMARY

An objective of the present invention is to provide a method for making an easy-to-view CT sheet, to resolve the problem of the existing method for making a CT sheet proposed in the background that may cause harm to a patient due to radiation dose during scanning.

To achieve the above purpose, the present invention provides the following technical solutions.

A method for making an easy-to-view computed tomography (CT) sheet is provided, where the method is implemented by the following modules: an X-ray tomographic scanning device, a computer system, an operating system, and a printing system, where: the X-ray tomographic scanning device is composed of an X-ray generator, an X-ray tube, and an X-ray detector, the X-ray generator of the X-ray tomographic scanning device is used for X-ray generation, the X-ray tube of the X-ray tomographic scanning device is used for X-ray production, and the X-ray detector of the X-ray tomographic scanning device is used for X-ray detection.

Preferably, the computer system is composed of a data acquisition system, a central processing system, a tape drive, and a console, the data acquisition system of the computer system is used for acquiring and sorting data, the central processing system of the computer system is used for analyzing and processing the data, the tape drive of the computer system is used for storing the data and subsequent backup, and the console of the computer system is used by an operator to set a program.

Preferably, the operating system is composed of a control terminal, an image display, and multiple camera devices, the control terminal of the operating system is used by the operator to control operation of the overall device, the image display of the operating system is used for viewing data of a table to be generated and subsequent optimization, and the multiple camera devices of the operating system are used for taking photos for diagnosis.

Preferably, the printing system is composed of a printer, a display screen, and a transmission device, the printer of the printing system is used for printing a CT sheet to be generated, the display screen of the printing system is used for observation and displaying, and the transmission device of the printing system is used for data transmission.

Preferably, operation steps of the X-ray tomographic scanning device are as follows:

step 1: first scanning with X-rays a layer with a particular thickness of a part of a human body, and saving and transmitting scanned data;

step 2: calculating scanned information to obtain an X-ray attenuation coefficient or an X-ray absorption coefficient of each voxel, arranging the obtained coefficients into a digital matrix, converting numbers in the digital matrix into small squares with different gray scales from black to white by using a digital/analog converter, and arranging the small squares into a matrix;

step 3: receiving, by the detector, an X-ray penetrating the layer, converting the X-ray into visible light, converting the visible light into an electrical signal through photoelectric conversion, converting the electrical signal into numerals by using an analog/digital converter, inputting the numerals into a computer for processing, and transmitting processed data;

step 4: transmitting the processed data to the inside of the image display, then performing a series of operations through the control terminal, arranging the data, and performing overall optimization on the data; and

step 5: transmitting the optimized data to the inside of the printer, and then making and printing a CT sheet by using the printer.

Preferably, operation steps of the printer are as follows:

step 1: in a process of using inkjet printing, first sequentially executing settings/printer commands in a start menu of a system; right-clicking a printer icon in a window that pops up subsequently; executing a properties command from a shortcut menu that opens; opening a detailed information tab page in a properties setting box that opens; and then selecting an option of “speed up printing during execution in the background” or “directly output to the printer” from a background printing settings item on this page, so that a printing speed can be effectively improved;

step 2: appropriately controlling a paper feeding distance, where if a sliding baffle on the left side of a paper feeding tray is too far away from a printing paper, the printing paper easily bends in a direction of the sliding baffle during feeding of the paper; or if the baffle is too close to the printing paper, the printing paper is squeezed with too much pressure, which increases a friction between the printing paper and the baffle, and hinders the paper feeding; therefore, the distance between the printing paper and the sliding baffle of the paper feeding tray needs to be appropriately controlled;

step 3: readjusting a position of a paper thickness adjustment lever according to a thickness of the printing paper until a fault is resolved; and

step 4: opening a main tab page in a printer properties window, selecting a standard mode, and then clicking an advanced button; and in a dialog box subsequently pops up, adjusting color balance, brightness, or other parameters according to an ink cartridge model and a media type that are used, or directly dragging a slider therein to reduce color print density, to save ink.

Preferably, the data acquisition system includes a data receiving module, a data saving module, and a data sending module, the data acquisition system is configured to save and send data generated after detection, and the data acquisition system is electrically connected to a power switch.

Compared with the prior art, the present invention provides a method for making an easy-to-view CT sheet, and has the following beneficial effects:

In the present invention, through increasing detection accuracy of a detector inside a scanning device, radiation dose can be effectively reduced, thereby avoiding large radiation hazards to a patient during scanning. In this way, image quality can be effectively improved without increasing the radiation dose or even when the radiation dose is reduced. In addition, a processing method for image reconstruction is further improved under joint action of multiple groups of X-rays, thereby further improving data generation, and resolving the problem of the existing method for making a CT sheet that may cause harm to a patient due to radiation dose during scanning.

### DETAILED DESCRIPTION

The present invention provides the following technical solution.

A method for making an easy-to-view CT sheet is provided. The method is implemented by the following modules, including an X-ray tomographic scanning device, a computer system, an operating system, and a printing system. The X-ray tomographic scanning device is composed of an X-ray generator, an X-ray tube, and an X-ray detector. The X-ray generator of the X-ray tomographic scanning device is used for X-ray generation. The X-ray tube of the X-ray tomographic scanning device is used for X-ray production. The X-ray detector of the X-ray tomographic scanning device is used for X-ray detection.

The computer system is composed of a data acquisition system, a central processing system, a tape drive, and a console. The data acquisition system of the computer system is used for acquiring and sorting data. The central processing system of the computer system is used for analyzing and processing the data. The tape drive of the computer system is used for storing the data and subsequent backup. The console of the computer system is used by an operator to set a program.

The operating system is composed of a control terminal, an image display, and multiple camera devices. The control terminal of the operating system is used by the operator to control operation of the overall device. The image display of the operating system is used for viewing data of a table to be generated and subsequent optimization. The multiple camera devices of the operating system are used for taking photos for diagnosis.

The printing system is composed of a printer, a display screen, and a transmission device. The printer of the printing system is used for printing a CT sheet to be generated. The display screen of the printing system is used for observation and displaying. The transmission device of the printing system is used for data transmission.

Operation steps of the X-ray tomographic scanning device are as follows:

Step 1: First scan with X-rays a layer with a particular thickness of a part of a human body, and save and transmit scanned data.

Step 2: Calculate scanned information to obtain an X-ray attenuation coefficient or an X-ray absorption coefficient of each voxel, arrange the obtained coefficients into a digital matrix, convert numbers in the digital matrix into small squares with different gray scales from black to white by using a digital/analog converter, and arrange the small squares into a matrix.

Step 3: Receive an X-ray penetrating the layer by using a detector, convert the X-ray into visible light, convert the visible light into an electrical signal through photoelectric conversion, convert the electrical signal into numerals by using an analog/digital converter, input the numerals into a computer for processing, and transmit processed data.

Step 4: Transmit the processed data to the inside of the image display, then perform a series of operations through the control terminal, arrange the data, and perform overall optimization on the data.

Step 5: Transmit the optimized data to the inside of the printer, and then make and print a CT sheet by using the printer.

Operation steps of the printer are as follows:

Step 1: In a process of using inkjet printing, first sequentially execute settings/printer commands in a start menu of a system; right-click a printer icon in a window that pops up subsequently; execute a properties command from a shortcut menu that opens; open a detailed information tab page in a properties setting box that opens; and then select an option of “speed up printing during execution in the background” or “directly output to the printer” from a background printing settings item on this page, so that a printing speed can be effectively improved.

Step 2: Appropriately control a paper feeding distance, where if a sliding baffle on the left side of a paper feeding tray is too far away from a printing paper, the printing paper easily bends in a direction of the sliding baffle during feeding of the paper; or if the baffle is too close to the printing paper, the printing paper is squeezed with too much pressure, which increases a friction between the printing paper and the baffle, and hinders the paper feeding; therefore, the distance between the printing paper and the sliding baffle of the paper feeding tray needs to be appropriately controlled.

Step 3: Readjust a position of a paper thickness adjustment lever according to a thickness of the printing paper until a fault is resolved.

Step 4: Open a main tab page in a printer properties window, select a standard mode, and then click an advanced button; and in a dialog box subsequently pops up, adjust color balance, brightness, or other parameters according to an ink cartridge model and a media type that are used, or directly drag a slider therein to reduce color print density, to save ink.

The data acquisition system includes a data receiving module, a data saving module, and a data sending module. The data acquisition system is configured to save and send data generated after detection. The data acquisition system is electrically connected to a power switch.

The working principle and application process of the present invention are as follows:

First, a layer with a particular thickness of a part of a human body is scanned with X-rays. An X-ray tube and an X-ray detector are separately mounted on two sides of a scanned tissue in opposite directions. When an X-ray generated by the tube passes through the scanned tissue, remaining rays passing through the tissue are received by the detector. When the X-ray penetrates a layer of the tissue including many voxels and is received by the detector on the opposite side, an X-ray amount has been attenuated a lot, which is a sum of X-ray attenuation values of all voxels in this direction. Then the X-ray tube rotates to a particular angle, and then sends out an X-ray along another direction. In this case, the detector on the opposite side can measure a sum of the X-ray attenuation values of all voxels along the second irradiation direction. X-ray scanning is repeatedly performed on the selected layer of the tissue for many times by using the same method in different directions, to obtain a sum of several X-ray attenuation values. As is highly sensitive to X-rays, the detector first converts a received X-ray into an analog signal, then converts the analog signal into a digital signal, and then inputs the digital signal to a central processing system of a computer. Scanned data is saved and transmitted. Scanned information is calculated to obtain an X-ray attenuation coefficient or an X-ray absorption coefficient of each voxel. Then the obtained coefficients are arranged into a matrix, and numbers in the digital matrix are converted into small squares with different gray scales from black to white, and are arranged into a matrix. The detector receives the X-ray penetrating the layer, and converts the X-ray into visible light. Then the visible light is converted into an electrical signal through photoelectric conversion, and the electrical signal is converted into numerals by using an analog/digital converter. The numerals are then input into a computer for processing, and processed data is transmitted. The processed data is transmitted to the inside of an image display, then a series of operations are performed through the control terminal, the data is arranged, and overall optimization is performed on the data. The optimized data is transmitted to the inside of a printer, and then a CT sheet is made and printed by using the printer.

Although embodiments of the present invention have been illustrated and described, it can be understood by a person of ordinary skill in the art that various changes, modifications, replacements, and variations may be made to the embodiments without departing from the principle and spirit of the present invention, and the scope of the present invention is subject to the appended claims and equivalents thereof.

1. A method for making an easy-to-view computed tomography (CT) sheet, wherein the method is implemented by a system comprising: an X-ray tomographic scanning device, a computer system, an operating system, and a printing system, the method comprising:
scanning with X-rays a layer with a particular thickness of a part of a human body using the X-ray tomographic scanning device, and saving and transmitting scanned data to the computer system;
calculating an X-ray attenuation coefficient or an X-ray absorption coefficient of each voxel from the scanned data using the computer system;
arranging the obtained coefficients into a digital matrix;
converting numbers in the digital matrix into gray scales values from black to white by using a digital/analog converter;
receiving, by the X-ray tomographic scanning device, an X-ray penetrating the layer;
converting the X-ray into visible light and converting the visible light into an electrical signal through photoelectric conversion;
converting the electrical signal into numerals by using an analog/digital converter;
inputting the numerals into the computer system to obtain processed data; and
transmitting the processed data.

2. The method for making an easy-to-view CT sheet according to claim 1, further comprising
transmitting the processed data to the inside of an image display of a control terminal; performing operations through the control terminal to arrange and optimize the processed data into optimized data, arranging the data; and
transmitting the optimized data to the inside of the printer system; and
printing a CT sheet showing the optimized data by using the printer system.

3. The method for making an easy-to-view CT sheet according to claim 2, wherein operation steps of the printer system comprise:
adjusting settings of the printer system to increase printing speeds;
appropriately controlling a paper feeding distance by adjusting the distance between a printing paper and a sliding baffle of a paper feeding tray of the printing system;
readjusting a position of a paper thickness adjustment lever of the printer system according to a thickness of the printing paper until a fault is resolved; and
adjusting color balance, brightness, or other parameters according to an ink cartridge model and a media type installed in the printer system to reduce color print density and save ink.

4. The method for making an easy-to-view CT sheet according to claim 1, wherein the computer system comprises a data receiving module, a data saving module, and a data sending module, wherein the computer system is configured to save and send data generated after detection, and the computer system is electrically connected to a power switch.
